(19) United States
(12) Patent Application Publication
(10) Pub. No.: US 2010/0324504 A1
(43) Pub. Date: Dec. 23, 2010

(54) PASSIVE FLOW REGULATOR FOR INFUSION OF MEDICAMENTS

(76) Inventors: Eric Chappel, Saint-Julien-en-Genevois (FR); Stephan Gamper, Lausanne (CH)

Correspondence Address: NIXON & VANDERHYE, PC, 901 NORTH GLEBE ROAD, 11TH FLOOR, ARLINGTON, VA, 22203, US

(21) Appl. No.: 12/866,840

(22) PCT Filed: Feb. 6, 2009
(86) PCT No.: PCT/EP09/51414

§ 371 Date: Aug. 9, 2010

(30) Foreign Application Priority Data
Feb. 9, 2008 (IB) 2008/050473

(51) Int. Cl. A61M 5/168; B23P 17/00

(52) U.S. Cl. 604/246; 29/890.09

(57) ABSTRACT

Flow regulator for the infusion of medicaments, comprising, in succession, a substrate (1), a channel (5), a spacer (3) and a membrane (4), the latter having at least one hole (6) communicating with the channel (5), characterized in that the regulator is produced from at least two separate elements (1-4), the first element comprising the membrane (4) and the second element comprising the spacer (3).

### FIELD OF THE INVENTION

The present invention pertains to the field of administering medicaments in liquid form. It relates more precisely to the flow regulators used for this purpose.

### PRIOR ART

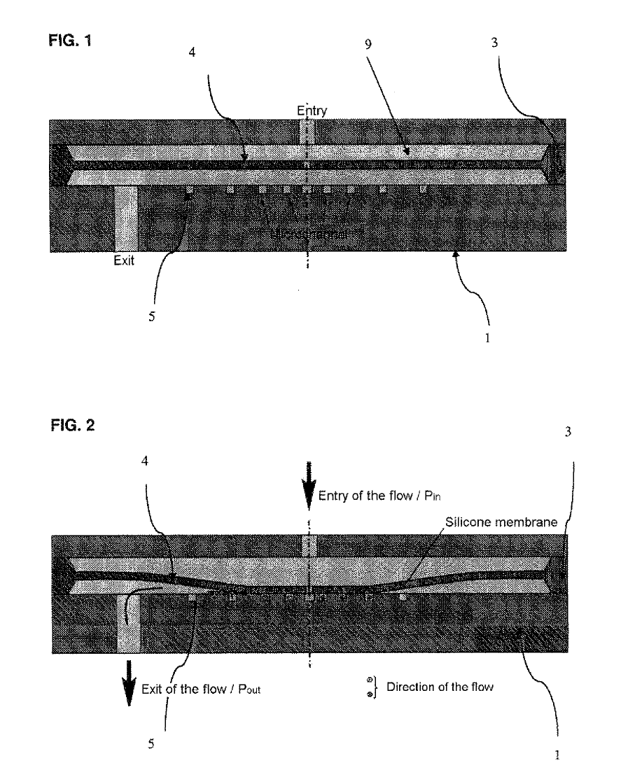

The operational principle of a flow regulator of the prior art (see FIGS. 1 and 2) is described in particular in the patent application WO 99/38552. It comprises a membrane 4 which is pierced at its center 6, is deflected by the pressure of the reservoir and enters into contact with a substrate 1 on which a fluidic channel 5 is etched, the latter being for example in the form of a spiral. The membrane 4 therefore closes this channel 5 after contact, except for the position where the central hole 6 is located. The liquid in the reservoir therefore flows through the central hole 6 then along the channel 5. The membrane 4, the distance between the membrane 4 and the substrate 1, and the channel 5, are dimensioned so that the fluidic resistance of the channel 5 varies proportionally with the pressure in the reservoir. The flow is therefore constant in a certain pressure range. It thus does not depend on the pressure. In the event of an overpressure, the channel 5 closes completely and the flow is interrupted.

The regulator described in the application WO 99/38552 is manufactured by etching, for example chemical etching, or by dry attack of the ion type, of the central hole 6 of the membrane 4, the membrane 4 itself, the spacer 3 between the membrane 4 and the substrate 1, and the channel 5 itself. The membrane 4 and the spacer 3 are therefore created simultaneously from the same element. An error in the thickness of the membrane 4 will thus also affect the thickness of the spacer 3, and vice versa.

### SUMMARY OF THE INVENTION

The present invention relates to a novel flow regulator structure and to a novel method, which simplifies assembly of the various parts of the regulator and makes it possible to achieve better manufacturing tolerances by virtue of the procedure employed.

The flow regulator according to the invention comprises in succession a substrate, a channel, a spacer and a membrane, the latter having at least one hole communicating with the channel. The regulator is characterized in that it is produced from at least two separate elements, the first element comprising the membrane and the second element comprising the spacer.

According to one embodiment of the invention, sheets with a controlled thickness and a low roughness are stacked, each sheet having a particular function. These sheets are obtained for example by rolling, then wire cutting or stamping, followed by mirror polishing. The stack is thus composed of a flat substrate with only one exit hole, a thin sheet with the through-channel, a thin sheet with a pierced disk for the spacer, and a thin sheet pierced at the center for the membrane. This stacking allows all the thicknesses to be controlled well before assembly, for example the membrane, the spacer and the depth of the channel, which is thus perfectly defined by the thickness of the sheet selected for this function. For a square shaped channel, the flow rate varies as the fourth power of the side length of this square, with the approximation of laminar flow for a Newtonian fluid at constant temperature. By this method, the tolerance for the depth is perfectly controlled and only the tolerance for the width of the channel remains, that is to say one over which there has already been good control.

The regulator according to the invention makes it possible to reach flow rate accuracies which could not be achieved by the standard methods of machining silicon or Pyrex.

The exit hole may also be offset toward the edge of the membrane in order to have the entry and the exit for the fluid on the same side.

The regulator according to the invention can close completely at a defined pressure in order to avoid overdoses. This threshold pressure is reached when the channel is fully covered by the membrane.

The regulator according to the invention may comprise means for measuring the deflection of the membrane, for example by using strain gauges placed in a Wheatstone bridge configuration on the membrane. These gauges may be produced by ion implantation or by diffusion, if the membrane is made of silicon. External gauges may also be placed on the membrane, for example by adhesive bonding, if said membrane is not made of a piezoresistive material.

### DETAILED DESCRIPTION OF THE INVENTION

The invention will be described in more detail below with the aid of examples, which are illustrated by the following figures:

FIG. 1, already described above, illustrates a flow regulator of the prior art in the neutral position.

FIG. 2, already described above, illustrates the regulator of FIG. 1 in active mode, when the membrane is deformed.

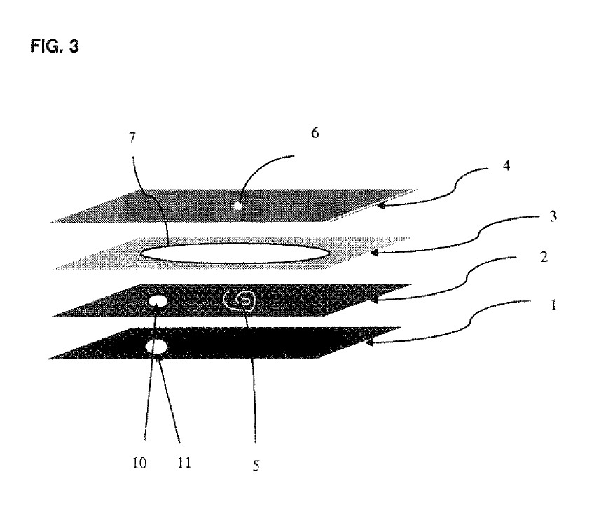

FIG. 3 presents an exploded schematic view of a first embodiment of a flow regulator according to the invention.

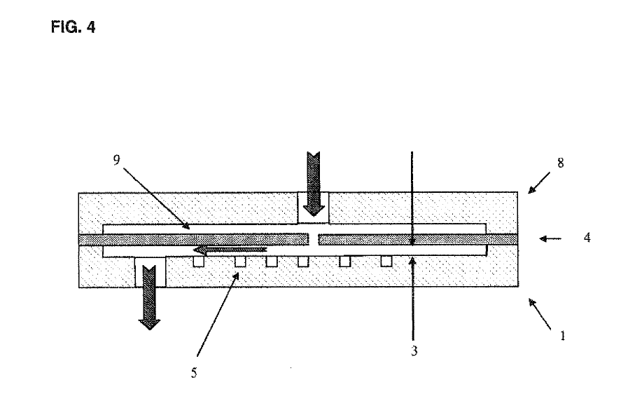

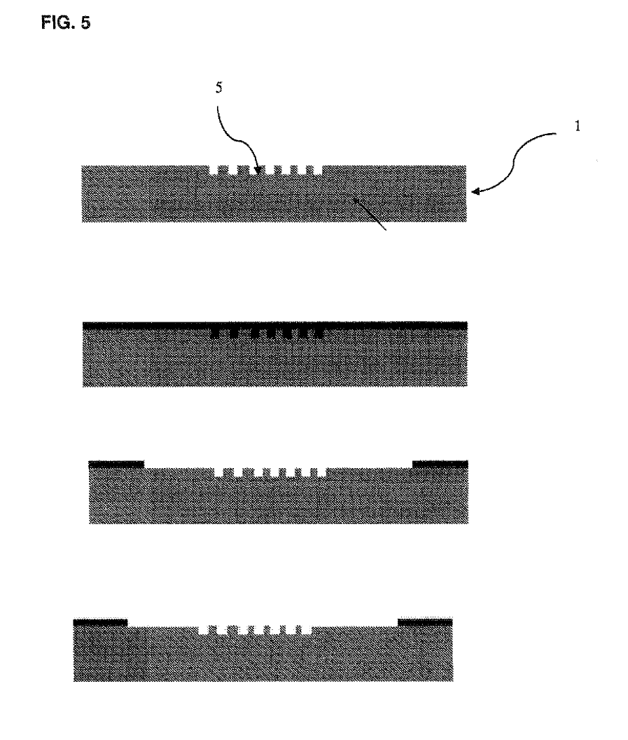

FIG. 4 presents a second embodiment of a flow regulator according to the invention.

FIG. 5 illustrates the steps of a method for producing a substrate-channel assembly according to the invention.

A first embodiment of the flow regulator according to the invention is illustrated in FIG. 3a. It is formed from four sheets 1 to 4, namely a first sheet intended to form the substrate, a second sheet 2 intended to contain the channel 5, a third sheet 3 comprising a large central opening 7 intended to form the spacer 3, and a fourth sheet acting as the membrane 4.

The membrane 4 may be made of polished silicon, that is to say a material which has excellent mechanical properties, but also of metal or any other material which has a high elastic limit. The membrane 4 may be pierced by chemical attack or by very short-pulse laser piercing, for example with a femtosecond laser, which avoids plastic deformations due to heating of the membrane 4. The direction of the laser piercing is important because there may be a ridge on the contour of the hole 6. A recessed circular part, the diameter of which is at least equal to the hole 6 of the membrane 4, is thus preferably formed at the center of the channel 5, which makes it possible on the one hand to increase the tolerances for positioning the membrane 4 relative to the channel 5 and also to prevent this possible ridge from creating a space and therefore poor sealing of the channel 5 at the hole 6.

The spacer 3, the channel 5 and the substrate 1 may preferably be made of metal, for example steel, or more advantageously of cofired ceramic LTCC. This is because these cofired ceramics are pierced, for example by a laser, machined, aligned, screen printed on the surface and then pressed in the green state, which allows three-dimensional stacking, and hot sintering subsequently allows permanent assembly of the various elements without a leak. The final assembly method, for example for rolled metal sheets, may be form fitting, welding or adhesive bonding.

For systems with large dimensions, it is preferable to use form fitting via two precision circular parts which enclose the various sheets, for example by clamping with the aid of screws. The upper part should be recessed in order to create a cavity 9 above the membrane and pierced at the center for the fluid inlet.

The fluid exit may be arranged in the lower part or in the upper part of the regulator.

When the fluid exit lies on the opposite side from the entry 6 (see FIG. 3), exit holes 10 and 11 will be arranged respectively in the sheet 2 containing the channel 5, as well as in the substrate 1.

When the exit lies on the same side as the entry, the membrane sheet 4 also comprises an outer hole with a larger diameter than the central hole for the exit.

Alignment is obtained either by additional centering holes, or by the substrate 1 itself. In fact, there is no tolerance for its thickness or for its width. It is easy to imagine a type of cavity into which the sheets are form fitted. Alignment pins may be used for systems with large dimensions. Alignment is facilitated if the contours of the sheets are circular, and so is the recess of the housing into which these sheets are form fitted.

The surface roughness of the membrane 4, the channel 5 and the substrate 1 must be very much less than the characteristic dimensions of the component, that is to say the depth of the channel 5, the thickness of the spacer 3, the thickness of the membrane 4 and the diameter of the entry hole 6.

The use of rolled sheets, which have been machined and already mirror polished before final assembly, allows the leaks during operation to be reduced substantially.

The channel 5 per se may be produced by electroplating directly on the substrate I, for example by forming the negative of the channel I by lithography in order to deposit the metallic base layer which, after growth, will define the channel 5 in relief. The negative part of the spacer 3 may also be produced by electroplating. The surface roughness obtained by electroplating is entirely compatible with correct operation of the component.

In order to maintain the principle of assembling a layer whose thickness is well controlled, the negative of the channel 5 may be grown on a sacrificial layer which will be dissolved before assembly of the substrate 1.

FIG. 4 represents a passive flow regulator assembly with the spacer 3, the channel 5 and the substrate 1 produced in a single part, for example by injection molding plastic material or ceramic, or alternatively by embossing plastic. The cap 8 may also be produced in this way. The cap 8 includes a cavity 9, which allows good distribution of the pressure above the membrane 4.

The membrane 4 is made of a material which has a high elastic limit and advantageously minimal internal stresses.

This method has numerous advantages over the machining methods of the prior art. The relative tolerances are wider with the method according to the invention, particularly for the micromachining of silicon or metal. When the substrate, the channel and the spacer are formed from a single element, the method according to the invention consists principally in producing a single mold with the required dimensions, for example by one or two nickel electroplating operations on a positive or negative of the channel and the spacer. Replication of the components by injection molding plastic or by embossing from this mold makes it possible to achieve excellent relative tolerances as well as a good surface roughness, by means of suitable process parameters such as cooling under pressure and the injection molding or embossing time. This method makes it possible to reduce considerably the manufacturing costs and the number of method steps.

A coating may be necessary on all the parts of the components in order to ensure their biocompatibility, for example a layer containing diamond, a layer of gold or a layer of titanium.

The various elements are subsequently assembled by direct plastic/silicon or plastic/metal bonding, or alternatively by adhesive bonding, form fitting.

A trench or a groove may be formed in the plastic or ceramic substrate in order to be able to spread the adhesive. The membrane 4 is then positioned on the substrate 1 for polymerization, optionally with an application pressure in order to maintain the assembly tolerances.

In the microreplication configuration, the difficulty reduces essentially to a single critical alignment, namely that of the membrane 4 on the replicated part. A technique of autocentering via a groove or an extra thickness on the replicated part may be used. Alignment holes may also be pierced in the membrane 4 and the replicated part of the regulator.

For fabrication methods involving microreplication, very particular attention must be paid to producing the model of the original part (master) which will subsequently be used to generate the injection molds or the embossing heads. The tolerances required for this element require that the master should be produced by micromachining techniques.
1. Wet etching, for example KOH, of a silicon wafer in order to produce the spacer then dry etching for the spiral, with a slight angle in order to facilitate mold release.
2. Dry or wet etching of Pyrex for the spiral and producing the spacer by depositing resin, for example SU8 by spinning, followed by photolithography and, optionally, finally metallization (see the process flow below).

FIG. 5 represents an exemplary embodiment of the master, including deposition of SU8 after dry etching of the channel, followed by photolithography and optionally metallization.

Of course, the invention is not limited to the embodiments which have been illustrated and discussed in the present text.

1. A flow regulator for the infusion of medicaments, comprising in succession a substrate (1), a channel (5), a spacer (3) and a membrane (4), the latter having at least one hole (6) communicating with the channel (5), characterized in that the regulator is produced from at least two separate elements (1-4), the first element comprising the membrane (4) and the second element comprising the spacer (3).

2. The flow regulator as claimed in claim 1, wherein the first element is a sheet.

3. The flow regulator as claimed in claim 1, wherein the second element also comprises the channel (5).

4. The flow regulator as claimed in claim 3, wherein the second element also comprises the substrate (1).

5. The flow regulator as claimed in claim 1, comprising a third element which comprises the substrate (1) and the channel (5).

6. The flow regulator as claimed in claim 1, comprising four elements (1-4), the first (4) comprising the membrane, the second (3) comprising the spacer, the third (2) comprising the channel and the fourth (1) comprising the substrate.

7. The flow regulator as claimed in claim 6, wherein the four elements (1-4) are sheets.

8. The flow regulator as claimed in claim 7, wherein at least one of the sheets (1-4) is obtained by rolling.

9. The flow regulator as claimed in claim 7, wherein at least one of the sheets (1-4) is made of polished silicon.

10. The flow regulator as claimed in claim 9, comprising means for measuring the deflection of the membrane (4).

11. The flow regulator as claimed claim 10, consisting of at least one sheet whose contour is circular.

12. The flow regulator as claimed in claim 11, in which the internal contour of the spacer (3) is circular.

13. The flow regulator as claimed in claim 12, dimensioned so that the fluidic path is obstructed when a threshold pressure of the fluid inside the regulator is reached.

14. A method for manufacturing a flow regulator as claimed in claim 1, comprising the stacking of four sheets (1-4), the first being intended to form the substrate (1), the second a channel (5), the third a spacer (3) and the fourth a membrane (4).

15. The method as claimed in claim 14 comprising a step of aligning the sheets (1-4).

16. The manufacturing method as claimed in claim 1, characterized in that the spacer (3) and/or the channel (5) are created by electroplating or any other deposition method.
